(19) United States
(12) Patent Application Publication
(10) Pub. No.: US 2021/0154278 A1
(43) Pub. Date: May 27, 2021

(54) IMMUNE GENE-DRUGS EXPRESSED IN TUMOR CELLS ACTIVATE THE SYSTEMIC IMMUNE RESPONSE AGAINST CANCER

(71) Applicant: SICHUAN ONCOCARE BIOPHARMACEUTICAL, INC., Chengdu (CN)

(72) Inventor: Li YU, Gaithersburg, MD (US)

(21) Appl. No.: 16/836,180

(22) Filed: Mar. 31, 2020

(30) Foreign Application Priority Data
Nov. 21, 2019 (CN) 201911149314.7

(51) Int. Cl. A61K 39/00; C12N 15/64; A61K 47/68; A61P 35/00

(52) U.S. Cl. CPC A61K 39/0011; C12N 15/64; A61P 35/00; A61K 47/6851; A61K 47/6803

(57) ABSTRACT

The disclosure discloses a group of therapeutic genes and their products, which activate specific immune T cells to elevate systemic immune response to treat solid cancer. The active ingredients of the immune gene-drugs are the human genes mainly encoding T cell-targeting molecules, so-called T cell costimulator. The expressed costimulators specifically activate a variety of T cells through receptor-ligand interaction to induce a systemic immune response. These therapeutic gene products, which normally produce in B lymphocytes, are grafted into tumor cells through vectors since the therapeutic effects of the gene drugs rely on the expressed functional proteins on the tumor cell surface. The transplantation of the therapeutic genes into the tumor is performed by DNA plasmids and/or replicable gene vectors that are viruses.

### CROSS-REFERENCE TO RELATED APPLICATION

This is a continuation application of a Chinese Application, which claims priority to Chinese Patent Application No. 201911149314.7, filed on Nov. 21, 2019, the entire contents of which are incorporated herein by reference.

### TECHNICAL FIELD

The present disclosure relates to the field of gene and immunotherapy of cancer.

### BACKGROUND

Immunotherapy of Cancer

Cancer is a serious threat to human health. According to the latest global cancer statistics, it is estimated that 18.1 million new cancer cases and 9.6 million deaths occurred worldwide in 2018. The statistical analysis by NIH NCI estimated 1,735,350 new cases of cancer were diagnosed in the United States and 609,640 people died from the disease in 2018. Furthermore, the number of new cancer cases is on the rise globally. The number of new cancer cases per year is expected to rise to 23.6 million by 2030. The number of global cancer deaths is projected to increase by 45% between 2008 and 2030. And deaths from cancer worldwide are projected to reach over 13 million in 2030. The most common cancers worldwide are: Lung (2.09 million cases); Breast (2.09 million cases); Colorectal (1.80 million cases); Prostate (1.28 million cases); Skin cancer (non-melanoma) (1.04 million cases); Stomach (1.03 million cases). The most common causes of cancer death are cancers of Lung (1.76 million deaths); Colorectal (862 000 deaths); Stomach (783 000 deaths); Liver (782 000 deaths); Breast (627 000 deaths).

Notably, the high mortality of cancer is due to mostly delaying early diagnoses and failing at available treatments with modern medical technology. Thus, the development of more effective medicine to treat cancer is very necessary to raise the level of human health and living quality. In recent years, immunotherapy of cancer has become a new battlefield against cancer. A variety of immunotherapy for cancer has been developed. Some were approved to apply in patients, including monoclonal antibodies (e.g. ipilimumab), chimeric antigen receptor-T cell therapy (CAR-T), and oncolytic viruses (e.g. T-Vec).

The immune system is the first defense to limit cancer development. Immune B and T cells are the main forces that recognize and clear cancers. B cells release antibodies and stimulate immune responses against cancer cells. Antigen-presenting cells (APCs) among B cells play key roles in initiating immune responses. APCs digest cancer cells and present their proteins (antigens) on their surfaces, helping other immune cells to recognize and destroy harmful cells. Also, APCs express T-cell costimulators to regulate and activate T-cell immune responses with the combination of WIC antigen complexes. CD4+helper T cells release a class of lymphocyte-activating factors (e.g. interleukins, chemokines) and send “help” signals to other immune cells, such as CD8+T cells, to mediate their response and to ensure destroying malignancy cells as quickly and efficiently as possible. Some CD4+T cells also communicate with antibody-producing B cells and preserve the memory of cancer antigens. CD8+T cell killers destroy infected cells and cancer cells. T-cells require two signalings for full activation. All T cells express homologous T cell receptors (TCR), which interacts with peptide-MHC molecules presented on the APC membrane to provide so-called the first signal for T cell activation. Whereas, the first signal is not enough to fully activate T cells and may also cause T cell anergy (T cell incompetence or low reactivity) and T cell deletion or immune tolerance. To fully activate T cells requires second signaling that is co-stimulation, which is produced in APCs. The two signals are necessary to stimulate T cell proliferation, differentiation, survival, and to induce system immune response (Chen, L. & Flies DB. Molecular mechanisms of T cell co-stimulation and co-inhibition. Nat Rev Immunol. 2013, 13: 227-242). Numerous studies have shown that the presence of effective co-stimulation is required for the immune system to eliminate tumors. Studies on T-cell costimulators has confirmed that T-cell costimulator vaccines or agonists (antibodies) of these factors can effectively inhibit tumor growth (Peggs, kS. et al. Cancer immunotherapy: co-stimulatory agonists and co-inhibitory antagonists. Clin Exp Immunol. 2009, 157: 9-19) (Benjamin, Y K, et al, On the Other Side: Manipulating the Immune Checkpoint Landscape of Dendritic Cells to Enhance Cancer Immunotherapy. Front. Oncol., 2019, 00050) (Gregory D., et al. Costimulatory and coinhibitory receptors in anti-tumor immunity. Immunol Rev. 2009. 229: 126-144).

Anergy T cells that have no immune response to tumor cells are usually present in the tumor microenvironment (Schwartz, R H. T cell anergy. Annu Rev Immunol. 2003, 21:305-34) (Brian T, Abe, et al. Uncovering the mechanisms that regulate tumor-induced T-cell anergy. Oncoimmunology. 2013, 2(2)). This leads tumors to grow without immune surveillance. Tumor cells generally hide or mutate their antigens to avoid attacking by the immune system; some cancer cells can produce inhibitory molecules to block the interaction of APC with T cells or inhibit T cells' attack on cancer cells. For example, research has demonstrated that nearly 70% of lymphoma constitutional produce inhibitory checkpoint CTLA-4 (cytotoxic T lymphocyte-related protein). In contrast to regulating T cell activation in lymphoid organs, CTLA-4 inhibits T cell function by binding to stimulus checkpoints CD80 and CD86 on APC cells and by neutralizing the function of CD28 receptors on T cells, and mainly affects naive T cells. Another type of inhibitory checkpoint PD-1 and programmed death-ligand 1 or 2 (PD-L1 or PD-L2) mainly down-regulates effector T cell activity in tissues and tumors. The lack of costimulatory signals produced by B cells in the tumor microenvironment also leads to the production of non-reactive T cells and therefore lacks an appropriate anti-tumor immune response.

Current immunotherapy of cancer mainly focused on removing inhibitory effects of checkpoint molecules, PD1/PDL1 and CTLA-4 to treat cancers. Monoclonal antibodies (Mabs) against these checkpoint inhibitors have been broadly administrated to cancer patients. However, immunotherapy with these Mabs has not obtained satisfactory efficacy. 5-year overall survival rate increases to 18% after Mab treatments (Zhongyuan F, et al. A meta-analysis of the efficacy and safety of PD-1/PD-L1 immune checkpoint inhibitors as treatments for metastatic bladder cancer. Onco. Targets. Ther. 2019, 12:1791-1801).

The development of more effective therapeutics is important to meet the needs in the treatment of cancer.

Altogether, the critical aspect in cancer immunotherapy is to awake anergy T cell-mediated immune response since B cells fail at recognizing the tumor antigens and incomplete to induce T cell activity.

### SUMMARY

The disclosure discloses using the immune gene products as therapy drugs that induce the immune system to treat cancer. The biological materials of immune gene-drugs are a piece or a fragment of human genes that encode particular immune modulators for activating immune T cells.

The active ingredients of the immune gene-drugs are T cell costimulatory molecules encoded by these genes, which are normally expressed in antigen-presenting cells. To treat cancer, these immune genes are grafted and expressed in tumor cells through DNA or virus vectors for therapeutic activity. The expressed T cell costimulators will mimic their counterparts produced in APCs to activate anergy T cells in the tumor microenvironment, thus to induce systemic immune responses against cancer.

These costimulators expressed in tumor surface will interact with T cells through specific ligands/receptors pathway resulting in a series of a chain reaction for T cell proliferation and differentiation and amplification of subset T cells and thus enhancement of immune system response to tumors. The induced systemic immune responses do not only destroy existing tumor cells but also can prevent tumor recurrence, which acts as like vaccination to prevent infectious diseases.

To effectively treat cancers, this disclosure provides the following technical solutions: (A) The immune gene products that activate systemic immune responses to tumors must be specifically targeting both, malignancy cells and immune T cells. (B) To meet the (A) requirement, the immune genes or gene fragments must be brought into tumor cells and express their T cell costimulators by vectors of either naked DNA plasmid or viruses.

Therefore, different from the CAR-T technique that activates the body immune system through transfection and/or transformation of the lymphocytes isolated from patients in vitro, this disclosure of activating the immune system takes place in vivo through vector-mediated gene transfer and expression. Thus, this kind of immune gene-drugs is composed of vector and immune genes and is directly administrated by intratumor injection.

Unlike those cancer immune therapies with monoclonal antibodies described above, of which the protein drugs were produced and manufactured in vitro, the immune gene-drugs described in this disclosure are constructed and manufactured in a form of naked DNA plasmids or/and virus vectors assembling the immune genes.

Different from other mechanisms in treating cancer, this disclosure of immune gene-drugs relies on activating specific T cells in the tumor microenvironment through “two signalings”, tumor antigens and expressed T cell costimulators in tumors.

Therefore, this disclosure of immune gene-drugs is completely different from other immune therapies of cancer in terms of the therapy materials (immune gene fragments); delivery route (in vivo expression); targeting cells (immune T cells); therapy mechanisms (two signalings).

### BRIEF DESCRIPTION OF THE DRAWINGS

The following drawings are merely examples for illustrative purposes according to various disclosed embodiments and are not intended to limit the scope of the present disclosure.

FIG. 1. Mouse studies show that the immune gene drugs inhibit tumor growth (top panels) and tissue section of the mouse tumors demonstrate the tumor cell death without directly touch the injected drugs.

FIG. 2. Tissue section of the mouse tumors demonstrates the massive lymphocyte invasion of tumor tissue caused by the drug-induced immune response.

FIG. 3. Both left- and right-side tumors with or without drug injection all show the repression or slow growth, comparing to PBS control.

FIG. 4. A variety of costimulators exhibit antitumor activity, whereas the PBS and IL2 group do not show such activity in bilateral tumor mice.

### DETAILED DESCRIPTION OF THE PREFERRED EMBODIMENT

The purpose of the present disclosure is to overcome the defects in the existing cancer immunotherapy and to provide novel pharmaceutics actively targeting the immune system in vivo to treat cancer.

The disclosure discloses a group of therapeutic genes and their products, which activate specific immune T cells to elevate systemic immune response to treat solid cancer. The active ingredients of the immune gene-drugs are the human genes mainly encoding T cell-targeting molecules, so-called T cell costimulator. The expressed costimulators specifically activate a variety of T cells through receptor-ligand interaction to induce a systemic immune response. These therapeutic gene products, which normally produce in B lymphocytes, are grafted into tumor cells through vectors since the therapeutic effects of the gene drugs rely on the expressed functional proteins on the tumor cell surface. The transplantation of the therapeutic genes into the tumor is performed by DNA plasmids and/or replicable gene vectors that are viruses. Therefore, the immune gene-drugs are composed of both the therapeutic genes and the vectors. They differ from other immune therapies of cancer through using specific immune genes that integrate into vectors and express in tumor cells to induce an immune response to cancers.

The immune gene-drugs described in this disclosure can directly induce the immune responses to destroy cancer cells and prevent cancer recurrent. These immune gene-drugs aim at T cell-mediated systemic immune response to recognize the malignancy and to kill cancer cells. This disclosure involves expressing T-cell costimulators in tumor cells and thus inducing the body's immune system to specifically fight the multiple cancers and/or metastasis which are failed at routine therapy by surgery, chimeric drugs, and radiation. Therefore, this disclosure is unique new immunotherapy, applying to treat unresectable solid cancers.

The disclosure discloses the application of a sort of immune-genes and their products, aiming specifically at T cell activation and thus enhancing a systemic immune response against cancer. The active ingredients of drugs mainly comprise of T cell co-stimulatory molecules. The features of these T cell costimulators include: They are the second signaling molecules required to stimulate anergy and/or naive T cells; they specifically interact with the subsets of T cells through ligand/receptor on a variety of T cells to induce a systemic immune response; they normally expressed in APCs instead of tumor cells.

These T cell costimulators originally expressed in APC cells and their different functions in T cell activation are listed in Table 1, including, but not limited to, CD80/86, ICOSL, OX40L, CD40, 4-1BBL, CD70, CD30L, and CD48. The amino acid sequences of these human-derived T cell costimulators used as immunotherapy cancer drugs are enclosed in this document below.

TABLE 1

| | | Expression Cells | | | |
|---|---|---|---|---|---|
| Type | Ligand:Receptor | APC | T cells | Tumor | Main Functions |
| Positive | B7-1/B72:CD28 | B7- | CD28 | | Activate anerge and naïve T cells; |
| Costimulators | | 1/B72 | | | enhance proliferation; produce |
| | | | | | cytokines. |
| | ICOS-L:ICOS | ICOS-L | ICOS | | Inducible expression for T cell |
| | | | | | proliferation and survival. |
| | CD40:CD40L | CD40 | CD40L | CD40 | Stimulate B cells; enhance MHCII |
| | | | | | expression. |
| | 4-1BBL:4-1BB | 4-1BBL | 4-1BB | | Stimulate CD4 and CD8 cells. |
| | OX40L:OX40 | OX40L | OX40 | | T cell proliferation and survival. |
| | Light:HVEM | Light | HVEM | | Induce T cell proliferation and |
| | | | | | survival; produce cytokines. |
| | CD70:CD27 | CD70 | CD27 | | T cell proliferation and survival. |
| | GITRL:GITR | GITRL | GITR | | Activate naïve T and Th cells; |
| | CD30:CD30L | CD30 | CD30L | | produce cytokines. |

Further, the human T cell costimulators encoded by these immune genes is a functionally active protein molecule or a protein polypeptide that can be modified or mutated as a plurality of homologous or heterologous fusion molecules. The functional activity of the human-derived T cell costimulators has a necessary second signal activity for activating T cells and a specific immune response to the tumor.

To activate the immune system against cancer, the inventor's strategy is to express these T cell costimulators in the tumors together with tumor antigens thus specifically activate T cells in the tumor microenvironment and to initiating systemic immune responses. The induced systemic immune responses should be tumor-tissue specific since both WIC I-antigen complexes of tumor cells and costimulators are presented to T cells through the two signalings.

These T cell costimulators target at the T cell subpopulation, including any one of CD4 cells, CD8 cells, NK cells, cytotoxic T cells, lymphokine T cells, inducible T cells, and helper T cells, respectively, and thus play key roles in immunotherapy of cancer through inducing systemic immune responses.

To effectively activate a variety of the anergy T cells in the tumor microenvironment, this disclosure takes a strategy that is expressing the T cell costimulators within tumors through vectors. To reach this goal, these T cell costimulator genes or gene fragments have to be covalently linked to a vector that contains a promoter, driving the therapeutic gene expression. The expression vectors can be any type of a deoxyribonucleotide or a ribonucleotide, for example, a simple plasmid DNA or replicable vectors such as viruses with the envelope packaging the DNA or RNA.

Further, if the viruses are used as a vector, it can be an attenuated or non-attenuated virus strain, a vaccine or non-vaccine strain with or without amino acid mutation or mutations at a non-coding region. No matter how the vector(s) is mutated or constructed, it can carry the human T cell costimulators and keep functions on immune activation.

Since there are several T cell costimulators and lymphocyte factors playing different roles in the activation of the immune system, alternatively integrating each of these active factors into the respect vector can greatly expand anti-cancer arsenal. The combination of these costimulators with a different activity to a subtype of T cells may obtain the best efficacy in cancer immunotherapy.

We have constructed the oncolytic flaviviruses vectors and DNA plasmid vectors that carry a variety of T cell costimulator and lymphocyte factor genes, respectively. These vectors with T cell costimulator genes have been tested in mouse tumor models and showed very promising results. The repression of 80% tumor growth was observed in bilateral tumors of a mouse, reflecting a systemic immune response, especially to the left side tumor that was not directly injected with drugs.

### Example 1

In example 1, the expression vector is the West Nile virus (WNV). A human T cell costimulator gene is inserted into the genome of WNV by conventional methods of genetic engineering without affecting the WNV self-replication.

The methods of genetic engineering include: PCR synthesizes the gene fragment of human or murine T cell costimulators or GFP gene fragment; the PCR synthesized gene fragments were ligated into WNV cDNA; the positive plasmid clones were determined by restriction mapping and sequencing and amplified in E. coli. The expression of the inserted the immune gene in the virus-infected cells was detected by immunofluorescence.

Efficacy assay of oncolytic viruses carrying T cell costimulator genes for treatment of lung cancer.

The mouse models with bilateral lung cancer were established by injection of 1×10⁶ of mouse LL2 cells at two-sided dorsal subcutaneous of 4-6-week-old female mice (C57). When growing tumors appeared about 80 mm³ in size, the gene drugs of WNV vectors carrying each T cell costimulator gene were injected only into the right-side tumor, respectively.

Control group: Inject 100 μl of PBS.

Experimental group A: Inject 100 ul of vector DNA containing WN/Mc86-1 (WNV virus carries full mouse B7 gene).

Experimental group B: Inject 100 ul of vector DNA containing 200 ug of WN/Mc86-2 (WNV virus carries a deleted mouse B7 gene)

Experimental group C: injection of 100 ul of vector DNA containing 200 ug of WN/MIcos-L (WNV virus carries mouse Icos-L gene)

Experimental group D: injection of 100 ul of vector DNA containing 200 ug of WN/Mc48 (WNV virus carries mouse CD48 gene)

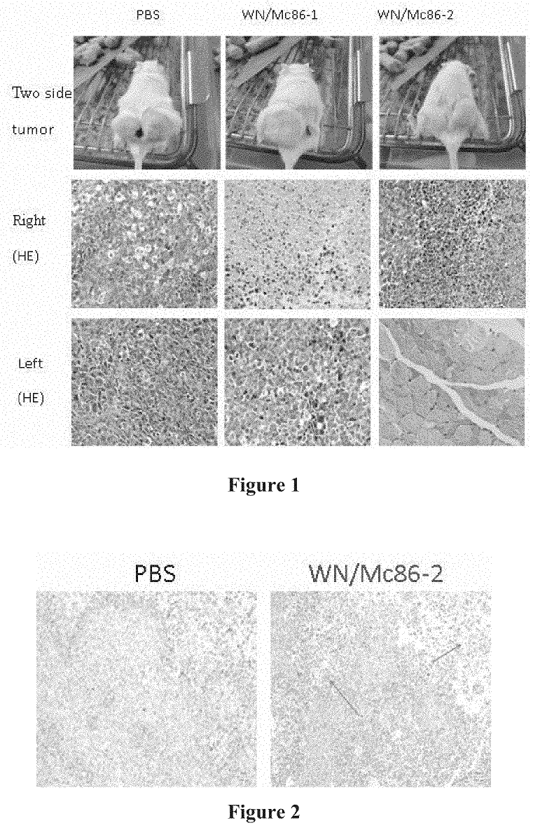

The mice were observed for 20 days after the administration and testing data were analyzed and shown in FIGS. 1 to 3.

(1) No symptoms were observed within 10 days after administration and no death occurred within 20 days to all testing mice.

(2) After the administration, the average tumor volume of the right tumors in the control group was 1309.85 mm³ and the average tumor volume of the left tumors was 1206.67 mm³.

In the experimental group A, the right tumor disappeared on the 7th day after the administration, whereas the growth of the left tumor did not stop, as the same as the PBS control group. In contrast to group A, both side tumors in group B showed a tumor to shrink or slow growth.

(3) The sections of tumor tissue were examined by immunohistochemistry and the results showed in FIG. 1.

Control group: All sections showed the malignancy characteristics: tumor cells were densely distributed with staining of cytoplasmic basophilic blue and heave nuclear chromatin; nuclei were not uniform in size and shape; typical images of pathological mitotic (asymmetry and multipolar mitotic).

Experimental group A: The tumor cells on the left were the same as showing in the control group. While, the right side of the disappeared tumor showed a large area of tumor cell necrosis; the nucleus was condensed, fragmented, lysed, and eosinophilic stained in the cytoplasm, indicating massive dead tumor cells.

Experimental group B: The tumor cells on the right also showed a portion of the necrosis area in tumor tissues, indicating partial death of tumor cells. While the shrink tumor tissue at the left side showed that all the tumor cells disappeared with the replacement of normal muscle cells.

(4) Immune staining of the tissue section of the left tumor in group B mouse showed—massive lymphocyte invasion, indicating immune response induced by the WN/Mc86-2 drug injected to the right tumor (FIG. 2).

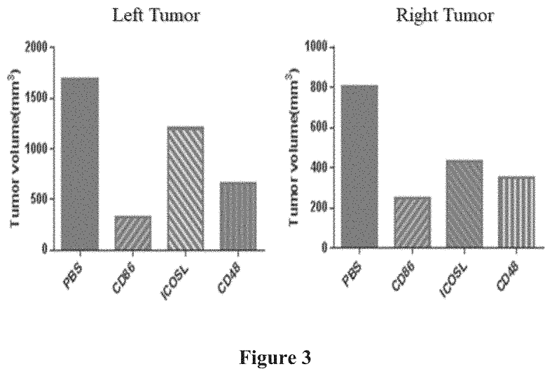

(5) Compared with the PBS control group, other experimental groups C & D administrated with immune gene B7 (CD86), Icos-L, and CD48, all showed significantly smaller tumor volumes at both the left and right sides, indicating inhibition of tumor growth by these immune gene products as showing in FIG. 3.

### Example 2

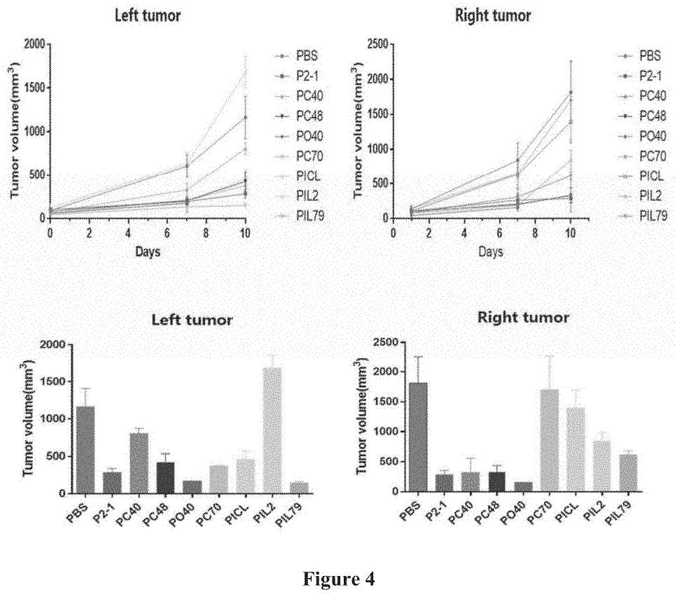

In the following example 2, the expression vector is DNA plasmid covalently connected with a variety of immune genes as labeled in FIG. 4. The procedures used to the construction of these plasmids are the same to construct the virus-carrying the immune gene.

The effects on immune suppression of tumors are evaluated with mouse tumor models too. The results showed that an average inhibition rate to both sides of tumors is more than 60%. The tumor sizes in the drug-treating group are significantly smaller than the PBS control group and smaller than the pIL2 group. The latter is an interleukin, a type of cytokine signaling molecule in the immune system but not belongs to the category of T cell costimulator. Therefore, these results prove that the specific activation of the immune system by the costimulators.

Conclusion of the immunotherapy for mouse lung cancer (LL2) as shown in FIG. 1-4:

WNV oncolytic virus carrying the immune genes as well as the DNA-immune gene plasmid shows dramatic inhibition effects on lung cancer growth. The repression of bilateral tumor growth is credited to the body's immune responses against cancer since all left-side tumors without drug injection have the same tumor repression rate as the tumors at the right side.

These animal studies demonstrate that the innovative immune gene-drugs can be a new weapon to win the battle against cancer diseases.

Also, sensitive mouse experiments confirm the safety of WNV oncolytic virus drugs. There is no evidence of neuroinvasive diseases and no morbidity has occurred when using these immune gene-drugs in cancer therapy.

What is claimed is:

1. An immune gene-drug, comprising:
therapeutic genes and vectors, of which active ingredients are immune modulators including T cell costimulators, encoded by immune genes; of which therapeutic gene products are ectopically expressed in tumors to specifically target immune T cells for activating and enhancing systemic immune response against malignant cells; of which the vectors carrying the immune gene and driving expression thereof are replicable or non-replicable DNAs or RNAs.

2. The immune gene-drug of claim 1, wherein encoded T cell costimulators play roles in specifically activating a variety of T cells.

3. The immune gene-drug of claim 1, wherein the therapeutic gene products and T cell costimulators are normally expressed in B lymphocytes or antigen-presenting cells (APCs) and are not expressed naturally in tumor cells.

4. The immune gene-drug of claim 2, wherein the T cell costimulator includes a group of T cell-activating factors that are CD80/86, ICOSL, OX40L, CD40, 4-1BBL, CD70, CD30L, and CD48, which has respectively correlating receptor/ligand on a variety of T cell surface.

5. The immune gene-drug of claim 4, wherein the T cell costimulators target and activate T cells through receptor-ligand interaction thus specifically induce different T cells to mediate immune responses.

6. The immune gene-drug of claim 4, wherein the T cell costimulators specifically activate a variety of T cell subsets, including CD4 cells, CD8 cells, NK cells, cytotoxic T cells, and some B cells, which play pivot roles in anti-cancer immunotherapy.

7. The immune gene-drug of claim 4, wherein the T cell costimulators are grafted into and expressed in tumor cells to execute induction of T lymphocyte activity in a tumor microenvironment.

8. The immune gene-drug of claim 4, wherein the T cell costimulators are expressed in a full or a part of a functional protein, or a mutated but functional protein or functional domain, and/or expressed in a fusion form of homologous or heterologous proteins.

9. The immune gene-drug of claim 8, wherein the expression of T cell costimulators is directed by a promoter that is integrated into the vectors.

10. The immune gene-drug of claim 4, wherein the T cell costimulators are carried into the tumor cells by vectors, which include DNA plasmid, DNA or RNA viruses, and vectors that bring the immune gene into tumor cells and drive the gene expression of functional proteins.

11. The immune gene-drug of claim 10, wherein the vectors are an attenuated or non-attenuated virus strain, a vaccine or non-vaccine strain, amino acid mutation or non-coding region mutation strain, or hybrid strains of viruses.

12. The immune gene-drug of claim 1, wherein the immune gene-drug is used in immunotherapy of solid cancer, which includes lung cancer, cervical cancer, lung epithelial cells cancer, prostate cancer, breast cancer, kidney cancer, colon cancer or epithelial cancers.
